(19) United States
(12) Patent Application Publication
(10) Pub. No.: US 2020/0375513 A1
(43) Pub. Date: Dec. 3, 2020

(54) PULSE OXIMETER SENSORS WITH REDUCED SENSITIVITY TO MOTION ARTIFACTS

(71) Applicants: Rajesh S. Kasbekar, Worcester, MA (US); Yitzhak Mendelson, Worcester, MA (US)

(72) Inventors: Rajesh S. Kasbekar, Worcester, MA (US); Yitzhak Mendelson, Worcester, MA (US)

(21) Appl. No.: 16/362,084

(22) Filed: Jun. 3, 2019

(51) Int. Cl. A61B 5/1455; A61B 5/00

(52) U.S. Cl. CPC A61B 5/14552; A61B 5/02416; A61B 5/6826; A61B 5/6824

(57) ABSTRACT

A reflective PPG sensor, in which motion artifacts have a reduced effect, is configured to be placed on one of a forehead, a finger or a wrist of a subject on a location selected according to a predetermined range of a signal quality index and has a force per unit area between 0.3 N/cm² and 1.5 N/cm², and a bonding pressure, for sensor to skin adhesion, between 10 N/cm² and 20 N/cm². In one instance, the reflective PPG sensor of these teachings also has a weight between 6 g and 12 g.

### BACKGROUND

This invention relates generally to pulse oximeter sensors with reduced sensitivity to motion artifacts.

Sensing technology for measuring vital signs such as SpO₂, pulse rate (PR), electro-cardiogram (ECG) and temperature, is increasingly transforming itself into mobile applications. This transformation is removing the barriers of time and space and enabling earlier detection of diseased states or monitoring of therapeutic effectiveness. Steady advances in sensors, hardware and wireless communication has led to the development of wearable mobile applications that has important implications on the quality of life.

Since its advent in the early 1980's, pulse oximetry has been used extensively for non-invasive monitoring of SpO₂ and PR in various applications including anesthesia, pulmonary function tests, bronchoscopy, intensive care, neonatal monitoring, sleep apnea studies, aviation medicine and monitoring oxygen therapy in the home. To understand the working behind pulse oximetry, let us look at the physiology of arterial oxygenation, hemoglobin saturation and the origin of the photo-plethysmograph (PPG) signal.

Oxygen is mixed with the blood in the lungs and is transported in blood mainly by hemoglobin. Ninety seven percent of the oxygen transported from the lung to the tissues is carried in chemical combination with hemoglobin in the red blood cells. The remaining three percent is transported in the dissolved state in the water of the plasma and blood cells. Chemically the oxygen molecule combines loosely and reversibly with the heme portion of the hemoglobin. The amount of oxygen that combines with hemoglobin is determined by the partial pressure of oxygen and can be depicted by the oxygen-hemoglobin dissociation curve. Because the blood leaving the lungs and entering the systemic arteries usually has a pressure of about 95 mmHg, the usual oxygen saturation of systemic arterial blood for a healthy adult averages 97 percent. In venous blood returning from peripheral tissues, the partial pressure of oxygen is 40 mm Hg and the oxygen saturation averages 75 percent. This oxygen saturation expressed as a percent of hemoglobin saturation is a measure of arterial oxygen (SaO₂) and is estimated by a pulse oximeter as a SpO₂ reading. Pulse oximetry is based on the difference in the selective absorption of red light by oxygenated blood or oxyhemoglobin (HbO₂) as compared to deoxyhemoglobin (Hb) or de-oxygenated blood. Despite a large variation in the optical absorption by biological tissues, blood exhibits similar optical absorption characteristics. This absorption can be differentiated into a constant, or time-invariant DC signal, and a pulsatile, or time-variant, AC signal caused by pulsating arterial blood. The reflected or transmitted light is converted into a voltage or current signal by the photodetector and is the origin of the PPG signal. Reflectance pulse oximetry uses the time-variant AC-PPG signal obtained by capturing reflected light (from two different wavelengths) using a photodetector and that is produced by changes in arterial blood volume associated with the pumping action of the heart. This technique also assumes that in ideal conditions there is no change in the non-pulsatile blood volume and that the amplitude of the arterial PPG pulse measured around a red wavelength (e.g. 660 nm) is sensitive to changes in arterial SpO₂. Taking these assumptions into consideration, SpO₂ can be derived by analyzing only the changes in the amplitude of the pulsatile red and infrared PPGs measured in the visible and near-infrared regions of the spectrum. Tissue absorption and non-pulsatile blood volume however can vary between individuals and probe locations. Furthermore, detected light intensities depend on detector sensitivity and incident light intensities. Therefore, a normalization process is commonly employed in which the pulsatile AC component of the red and infrared PPGs measured by a photodetector (PD), which results from the influx of arterial blood and expansion of the cutaneous vascular bed, is divided by the corresponding non-pulsatile DC component of the PPG that is characteristic of the background light absorbed by the tissue, the residual non-pulsatile arterial blood present in the vascular bed during diastole, and venous blood. This effective scaling yields a normalized AC/DC ratio for both the red and infrared wavelengths. The ratio of these two normalized AC/DC ratios, which is largely independent of the incident light intensity, is denoted by R and is equal to [(ACr/DCr)]/[(ACir/DCir)], where ACr is the pulsatile red component, ACir is the pulsatile infrared component, DCr is the non-pulsatile red component and DCir is the non-pulsatile infrared component of the corresponding PPGs. It is this adjusted R ratio that the pulse oximeter uses to compute SpO₂.

Pulse Oximetry can be performed in either transmission or reflectance modes. In transmission mode, the light emitting diodes (LEDs) and (PD) are mounted on opposite sides of a peripheral pulsating vascular bed across a finger-tip, foot, or ear lobe. Flexible bandage type disposable sensors or reusable finger clip-on probes are most commonly used as transmission type sensors.

In contrast to transmission mode, in reflectance pulse oximetry, reflected light rather than transillumination is used. The LEDs and PD are mounted side by side to enable readings from a planar vascular bed that has arterial, venous and capillary vessels. Reflectance pulse oximetry can often be more effective as compared to transmission pulse oximetry in medical applications where peripheral perfusion is compromised. In addition, the advantage of a reflectance oximeter is that it can monitor SpO₂ at various locations on the body including more central locations that are not accessible via conventional transmission mode oximetry. The most compelling reason to use reflectance type sensors is the ability to measure SpO₂ from alternate locations such as the forehead. The forehead is an optimal surface for reflectance pulse oximetry due to its planar surface and good blood perfusion. Pulse oximetry measurements from the forehead offer a potential advantage in mobile settings that require unimpeded use of the hands that can introduce excessive motion artifacts, such as, while monitoring soldiers on a battlefield. In applications such as sports or during sleep, Bideaux et al. (Bideaux, B., Schwendemann, T., Hey, S., Evaluation of design parameters for a reflection based long-term pulse oximeter sensor, Mobihealth: London, Great Britain, 2015) found that reflectance sensors did not interfere with the subject's activity, while transmission mode sensors were found to be uncomfortable and confining. Bideaux et al. also conducted market research which showed that reflectance mode sensors were predominantly used over transmission-based sensors in applications where the sensor was used to monitor blood oxygen level over a long period of time such as several hours or days. Furthermore, manufacturers of optical-based heart rate monitors are increasingly using these reflectance-based sensors in smart watches or other wearables such as wrist monitors, temple-based headband monitors (Moov™ Fitness Tracker) as well as ear bud monitors (BOSE Soundsport® Pulse or the SAMSUNG Gear IconX). If feasible, some manufacturers would like to expand the applications of similar wearable devices to include SpO₂ and associated pulse rate measurements. For these reasons, embodiments in which the sensor design was used for SpO₂ measurements were targeted for a reflection-based pulse oximetry application.

Pulse oximeters were originally used in the operating room on anesthetized patients. However, pulse oximeters are increasingly being used in general wards and wearables where patients can be free to move around. Pulse oximeters detect a pulsatile AC signal that is normally a very small fraction (1 to 3%) of the non-pulsatile DC component in the PPG. Therefore, motion in general and especially any transient motion of the sensor relative to the skin that is usually accompanied by changes in optical coupling between the sensor probe and the illuminated tissue can cause significant artifacts. The tolerance of pulse oximeters to motion artifacts has emerged as the most challenging technical problem and has been cited by clinicians repeatedly as the most common cause of false alarms (see Petterson, M. T.; Begnoche, V. L.; Graybeal, J. M., The effect of motion on pulse oximetry and its clinical significance. Anesth Analg 2007, 105 (6 Suppl), S78-84).

### Effect of Motion Artifact on Mobile Reflectance Pulse Oximetry

Motion artifact can have a significant impact on pulse oximetry. Petterson et al. discussed two models by which motion artifact could cause poor signal quality and false alarms. The first model assumes that noise due to varying pathlengths between the red and infrared signals induced due to motion is equally superimposed on the red and infrared signals. Therefore, as noise increases, the R ratio approaches 1, corresponding to a constant SpO₂ reading typically about 85%. The second model assumes low oxygen blood sloshing in the veins resulting in local perturbation of the venous blood that adds to the variable component of the PPG signal, making the device unable to distinguish between arterial and venous pulsations. The relative motion of the sensor with respect to the skin and the contact force in combination with the sensor weight can be used to explain the varying path length model described above. Similarly, placement would affect the signal-to-noise ratio based on exposure of the photodetector to venous pulsations. The source of motion artifact would therefore depend on factors such as sensor weight, relative motion of the sensor with respect to the skin, formation of air gaps between the sensor and skin during physical activity, contact force changes between the sensor and the skin and the location of the sensor on the body site due to inhomogeneous surface properties of the skin. These sources of motion artifacts affect the bias as well as variation of the PPG signal and signal-to-noise ratio.

### Current Methods for Mitigating Motion Artifact Based on Single Factor Methods

Current investigational techniques have focused on software-based mitigation strategies such as computational algorithms that have attempted to isolate the effects of undesired motion induced artifacts by rejecting suspect estimates of signal values. A classic example involves the use of adaptive noise cancellation that extracts the signal and removes the in-band noise using a signal extraction algorithm [SET®] developed by MASIMO. Other signal processing techniques have used accelerometers to act as a motion reference for removing motion artifacts (see, for example, Karantonis, D. M.; Narayanan, M. R.; Mathie, M.; Lovell, N. H.; Celler, B. G., Implementation of a real-time human movement classifier using a triaxial accelerometer for ambulatory monitoring. IEEE Trans Inf Technol Biomed 2006, 10 (1), 156-67). In addition, various filtering algorithms based on classification techniques using support vector machine (see, for example, Chong, J. W.; Dao, D. K.; Salehizadeh, S. M.; McManus, D. D.; Darling, C. E.; Chon, K. H.; Mendelson, Y., Photoplethysmograph signal reconstruction based on a novel hybrid motion artifact detection-reduction approach. Part I: Motion and noise artifact detection. Ann Biomed Eng 2014, 42 (11), 2238-50), reconstruction of motion artifact corrupted SpO₂ and PR signals based on time-varying spectral analysis (Salehizadeh, S. M.; Dao, D.; Bolkhovsky, J.; Cho, C.; Mendelson, Y.; Chon, K. H., A novel time-varying spectral filtering algorithm for reconstruction of motion artifact corrupted heart rate signals during intense physical activities using a wearable photoplethysmogram sensor. Sensors (Basel) 2015, 16 (1)) and iterative motion artifact removal using singular spectral analysis (Salehizadeh, S. M.; Dao, D. K.; Chong, J. W.; McManus, D.; Darling, C.; Mendelson, Y.; Chon, K. H., Photoplethysmograph signal reconstruction based on a novel motion artifact detection-reduction approach. Part II: Motion and noise artifact removal. Ann Biomed Eng 2014, 42 (11), 2251-63, Foo, J. Y.; Wilson, S. J., A computational system to optimise noise rejection in photoplethysmography signals during motion or poor perfusion states. Med Biol Eng Comput 2006, 44 (1-2), 140-5) during physical activities have improved the accuracy, sensitivity and specificity of SpO₂ and PR estimations. Several other techniques based on principle component analysis (PCA), reference signal reconstruction, independent component analysis (ICA), and non-linear artifact reduction (NLAR) have shown promising results.

Despite the extensive efforts to mitigate the effects of motion artifacts in pulse oximetry by employing advanced computational techniques, to optimize SpO₂ and PR estimations, the artifacts in the ‘acquired signal’ need to be minimized utilizing a sensor that is immune to motion. None of these reconstruction or computational methods consider the factors that reduce artifacts in the signal acquisition process due to motion. These methods use signals that are already adulterated with motion and noise artifact due to limitations in the sensor probe. Then they apply signal processing, filtering and reconstruction techniques to extract and reconstruct the PPG signals attempting to improve measurement accuracy. However, there are limitations in the processing of PPG signals with an inherent high level of motion artifact in the ‘acquired signal’.

Recently, several hardware approaches to address motion artifact have been applied to decrease the interference through optimal sensor design (see Salehizadeh, S. M.; Dao, D.; Bolkhovsky, J.; Cho, C.; Mendelson, Y.; Chon, K. H., A novel time-varying spectral filtering algorithm for reconstruction of motion artifact corrupted heart rate signals during intense physical activities using a wearable photoplethysmogram sensor. Sensors (Basel) 2015, 16 (1), Mendelson, Y.; Duckworth, R. J.; Comtois, G., A wearable reflectance pulse oximeter for remote physiological monitoring. Conf Proc IEEE Eng Med Biol Soc 2006, 1, 912-5, Warren, K. M.; Harvey, J. R.; Chon, K. H.; Mendelson, Y., Improving pulse rate measurements during random motion using a wearable multichannel reflectance photoplethysmograph. Sensors (Basel) 2016, 16 (3)). These sensor designs have been investigated using only one factor at a time. Mendelson et al (in Mendelson, Y., Dao, D, K, Chon, K, H. In Multi-channel pulse oximetry for wearable physiological monitoring., IEEE, 2013; 1231-123) studied the effect of placement using six reflectance sensors in a multi-channel wearable sensor configuration. These investigators concluded that PPG signals obtained from the different placements of each sensor on the forehead were affected differently by motion artifacts. Warren et al further investigated the use of a multichannel estimate using a template matching algorithm that chose the sensor with the least motion artifact and concluded that such a multi-channel estimate outperformed the other PPG sensors that were less tolerant of motion artifact. Yamaya et al (Yamaya, Y.; Bogaard, H. J.; Wagner, P. D.; Niizeki, K.; Hopkins, S. R., Validity of pulse oximetry during maximal exercise in normoxia, hypoxia, and hyperoxia. J Appl Physiol (1985) 2002, 92 (1), 162-8) concluded that sensor location significantly improved the performance of the forehead pulse oximeter sensor. These findings validate the importance of placement as one important variable in mitigating motion artifacts. Other ‘single variable’ investigations used the relative motion of the sensor as well as contact force between the sensor and the skin. Agashe et al (Agashe, G. S.; Coakley, J.; Mannheimer, P. D., Forehead pulse oximetry: Headband use helps alleviate false low readings likely related to venous pulsation artifact. Anesthesiology 2006, 105 (6), 1111-6) concluded that a contact force of 20 mmHg (0.27 N/cm²) on the forehead sensor using an elastic headband significantly improved SpO₂ errors and provided consistent performance by reducing artifacts due to venous pooling and pulsations. These variables, however, were studied independently of each other. Due to the interdependencies of one or several of these variables with each other, these approaches may have resulted in sub-optimal solutions to minimize the effects of motion artifact during signal acquisition.

Bideaux et al investigated the AC/DC ratio and signal-to-noise ratio of reflection type pulse oximeter sensors on the wrist using different design parameters such as contact pressure and the distance between the photodiode and the red and infrared LEDs. Bideaux et al concluded that constraints related to a minimum contact pressure of 15 mmHg (or 0.2 N/cm²) and motion artifacts were major impediments in the creation of a commercial product. Despite these constraints, they observed an AC/DC ratio of 1-3% at 45 mmHg or 0.6 N/cm² and a signal-to-noise ratio larger than 16 dB in the absence of motion artifact. They did not, however, evaluate the cumulative effect of any sensor variables on motion artifact or suggest ways to minimize it.

Current methods have used a single factor approach in sensor design to mitigate motion artifact. Several of these methods have been elaborated in the discussion section above. This single factor approach can often lead to wrong conclusions due to a failure to consider multi-factor interactions.

There is a need for pulse oximeter sensors with reduced sensitivity to motion artifacts.

### SUMMARY

Pulse oximeter sensors with reduced sensitivity to motion artifacts are disclosed herein below.

In one or more embodiments, a reflective PPG sensor of these teachings, in which motion artifacts have a reduced effect, is configured to be placed on one of a forehead, a finger or a wrist of a subject on a location selected such that a signal quality index has a value in a predetermined range of a signal quality index values and the reflective PPG sensor has a force per unit area between 0.3 N/cm² and 1.5 N/cm², and a bonding pressure, for sensor to skin adhesion, between 10 N/cm² and 20 N/cm².

In one instance, the reflective PPG sensor of these teachings also has a weight between 6 g and 12 g.

In one or more embodiments, a method of these teachings for selecting a reflective PPG sensor in which motion artifacts have a reduced effect includes selecting a placement of the reflective PPG sensor on a subject, selecting a contact force, and selecting a sensor to skin adhesion and contact force combination, such that motion artifacts have a reduced effect. In one instance, the method of these teachings also includes selecting a sensor weight and sensor to skin adhesion combination such that motion artifacts have a reduced effect.

Other embodiments are also disclosed hereinbelow.

For a better understanding of the present teachings, together with other and further objects thereof, reference is made to the accompanying drawings and detailed description and its scope will be pointed out in the appended claims.

### BRIEF DESCRIPTION OF THE DRAWINGS

FIG. 1 is shows a block diagram of one embodiment of a test prototype sensor used in these teachings;

FIG. 2a shows one embodiment of the test prototype sensor;

FIG. 2b shows a block diagram of one embodiment of the evaluation board used in the test prototype sensor;

FIG. 2c shows one embodiment of an optical module used in the test prototype sensor;

FIG. 3a shows the normality and independence of the data for test results from one embodiment of these teachings;

FIG. 3b shows a PPG signal without motion obtained in one experimental run of these teachings; and

FIG. 3c represents a PPG signal with motion artifacts obtained in one experimental run of these teachings.

### DETAILED DESCRIPTION

The description is not to be taken in a limiting sense, but is made merely for the purpose of illustrating the general principles of these teachings, since the scope of these teachings is best defined by the appended claims.

The above illustrative and further embodiments are described below in conjunction with the following drawings, where specifically numbered components are described and will be appreciated to be thus described in all figures of the disclosure:

As used herein, the singular forms “a,” “an,” and “the” include the plural reference unless the context clearly dictates otherwise.

In these teachings, the effect of several variables such as sensor weight, relative motion, placement and contact force against the skin that can impact motion artifact independently or by interacting with each other have been investigated. The dependencies and interdependencies of these key sensor variables on the acquisition of the PPG that will minimize the motion artifact signal. The four key sensor variables explored include sensor weight, relative motion against the skin controlled through use of an adhesive tape, contact force against the skin and placement on the body. These factors have an independent as well as a combined effect, in some instances, in reducing the effect of motion artifact. The factors or the interactions that have a maximum impact in reducing the motion artifact signal were identified, thereby increasing the signal-to-noise ratio. When this transduced signal is used in combination with the signal processing algorithms mentioned above, a maximum reduction in motion artifact can be achieved. Through a carefully designed full factorial experiment, the difference in the ratios of red to infrared PPG signals without motion and with motion can be minimized by identifying and selecting factors that have a significant impact in this minimization directly or due to interaction with other factors. A unique combination of these variables that is most optimal in reducing motion artifacts has been identified using a full factorial design of experiments methodology and the effect of these factors on PPG readings with and without motion has been evaluated.

### Multi-Factorial Design

The experiment consisted of a multi-factorial design consisting of a total of four factors at two levels with two replicates on a sample size of ten subjects with adequate representation of males and females.

A full factorial design for the four factors resulted in 16 experimental runs for the whole experiment and a total of 32 runs with two separate repetitions. (In some instances, a fractional factorial design could be used.) For a sample size of 10 subjects, this consists of a total of (16 factor combinations×2 repetitions×10 subjects) 320 readings with and without motion. This sample size had enough power to draw a reasonable conclusion.

### Factor Identification

The experimental protocol included the final four factors consisting of sensor weight, relative motion against the skin (sensor-to-skin adhesion), placement on the human body (forehead versus finger) and applied force (gentle versus firm).

Sensor placement and contact force have a significant impact on motion artifact and, therefore, were not part of the screening design (se, for example, Dresher, R. P.; Mendelson, Y., Reflectance forehead pulse oximetry: effects of contact pressure during walking. Conf Proc IEEE Eng Med Biol Soc 2006, 1, 3529-32; Agashe, G. S.; Coakley, J.; Mannheimer, P. D., Forehead pulse oximetry: Headband use helps alleviate false low readings likely related to venous pulsation artifact. Anesthesiology 2006, 105 (6), 1111-6; Teng, X. F.; Zhang, Y. T., The effect of contacting force on photoplethysmographic signals. Physiol Meas 2004, 25 (5), 1323-35; which are incorporated by reference herein in their entirety and for all purposes). Herein below, the Plackett-Burman design was used to identify the remaining factors, namely sensor size, weight and relative motion against the skin that were then used in the full-factorial design. (For a definition of Plackett-Burman designs, see, for example, Stephen R Schmidt, Robert G Launsby, Understanding Industrial Designed Experiments, Air Academy Press, Colorado Springs Colo., 1994, pp. 3-21 to 3-24.) The results from these initial experiments were used to make the final selection of the factors that were used in the full factorial experimental model. The final factors that have been identified based on these initial experiments were sensor weight, relative motion against the skin that was controlled by sensor-to-skin adhesion, contact force against the skin and different placements on the body (i.e., finger using reflectance pulse oximetry versus forehead using reflectance pulse oximetry). Optical density was not considered as a factor because a current was used that gave the best signal quality based on an empirical assessment of the signal morphology. This eliminated the need to find an alternate density that would be more optimal.

In one instance, the two levels for sensor weight included a low weight of 8.44 g and a high weight of 18.2 g. These weights were chosen based on the present assessment of commonly available pulse oximetry sensors, because these commercial sensors typically weigh between 6 g for flexible bandage type sensors and 22 g for finger clip-on sensors that are integrated with the device. The selection of the low and high values chosen above for the weight variable gave enough contrast and were within the weight range of commercial sensors. Sensor to skin adhesion was investigated using a two-sided adhesive tape, and the placement was varied between the forehead and finger. The finger represents an alternative measurement site with a different optical penetration depth and path length for the red and infrared light compared to the much thinner skin and subcutaneous tissue layers on the forehead and was, therefore, used as a contrasting factor level for placement investigation. The fingers are also subjected to a much higher level of motion in mobile applications compared to the forehead. Therefore, the finger and forehead were chose in the present model instead of using two alternate forehead sites. The finger was used instead of the wrist due to the higher PPG signal that can be obtained from the finger that is enhanced by the additional optical reflection from the skeletal bone (phalanx) as well as a denser and more well perfused vascular bed compared to the wrist. Both the forehead and the finger measurements were collected with the same reflectance sensor to ensure that no bias is introduced due to different sensor characteristics. Applied force included a low force of 0.5N that caressed the skin and a high force of 5N that slightly squeezed the superficial blood vessels. These values were determined experimentally. A force of 0.5N gave high AC amplitudes and high AC to DC ratios providing the most optimal PPG signals. A high value of 5 N was chosen as the upwardly means to study the effect of a significantly higher force a motion artifact tolerance by reducing venous pulsations and relative motion between the tissue and probe.

### Test Prototype

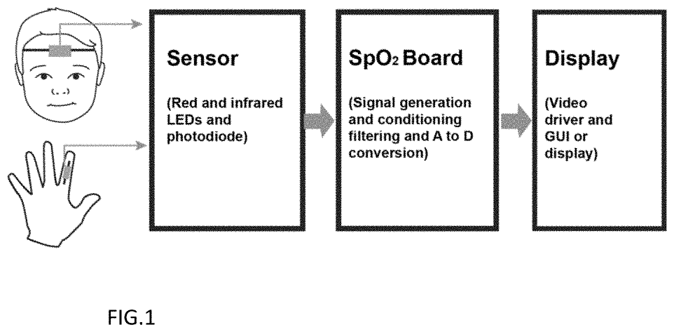

A prototype wearable reflectance type pulse oximeter sensor was developed to investigate the effect of motion artifacts on different sensor configurations and factors. FIG. 1 shows the experimental setup of the sensors, interfacing hardware and graphical user interface display.

The DCM03 optical module sensor manufactured by APM Korea consisting of red (660 nm) and infrared (905 nm) LEDs and a photodiode was used to measure the reflected light. The pulse oximeter was powered by the Texas Instruments (TI) AFE4490 evaluation board, which was used to drive the LEDs, read the output of the photodiode and perform the signal conditioning, filtering and analog to digital conversion. The AFE4450 is the integrated circuit (IC) specifically designed for pulse oximetry that is used in the AFE4490 pulse oximetry evaluation system board. TI also provided the associated Graphical User Interface (GUI) software [AFE4403EVM GUI] to connect the evaluation board to a personal computer and capture the waveform and digital data. The waveforms and digital values are displayed by the Graphical interface, as shown in FIG. 1.

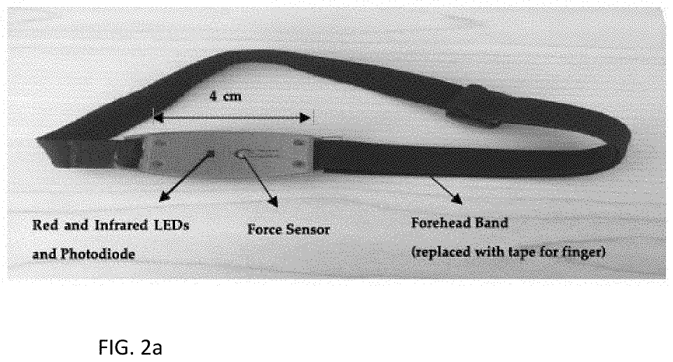

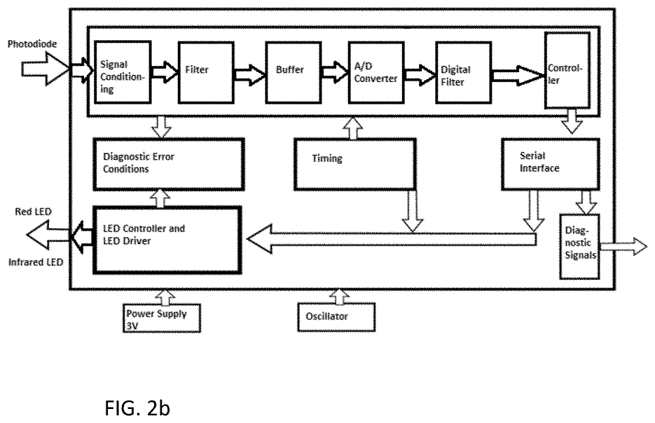

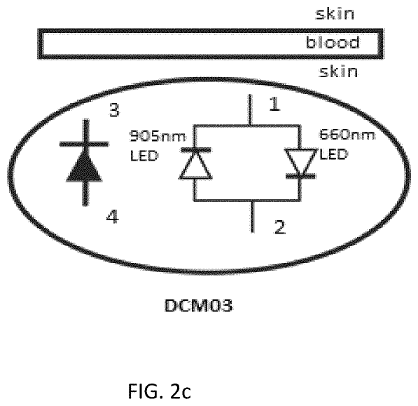

The sensor is shown in FIG. 2a and can be used on the forehead as well as the finger with the only exception being that the forehead band was replaced with a tape to attach the sensor on the finger. The AFE4490 Texas Instruments development board along with the front end DCM03 Optical Module (See Texas Instruments' Miniaturized Pulse Oximeter Reference Design, TIDA-00311, 2014) are shown in FIGS. 2b and 2c, respectively.

A Honeywell FSS005WNGB force sensor utilizing a custom developed instrumentation amplifier was used to measure the external force applied by the sensor housing against the skin. The ball shaped force sensor was placed just inside a small rounded protrusion emanating from the flat sensor surface as shown in FIG. 2a. When pressed against the skin by the headband or finger, the force sensor produced a signal that was measured using an oscilloscope and was calibrated by a commercial force meter. The pulse oximetry board was initialized using the initialization parameters specified by Texas Instrument. A current of 21 mA was used to activate the red and infrared LEDs (in accordance with the LED specifications) throughout all the experimental runs.

### Evaluation Procedure

Data were collected on ten healthy volunteers between the ages of 10 and 60. Of the ten participants, four were females and six were males. Four subjects had dark skin pigmentation and six had light pigmentation. To analyze the effect of motion on different sensor configurations without introducing any bias, data were collected from all subjects in an upright sitting position.

All tests were approved by the local IRB. Informed consent was taken for all adults along with an assent form for one child. The subjects were first asked to rest for a few minutes and baseline data were recorded with no motion. Two repetitions were completed for each experimental run. Red and infrared PPG signals obtained from the experimental set up were recorded with and without motion. The photodiode peak, mean and root mean square currents were averaged over a window and calculated by the software. The AC/DC current ratios for the red and infrared wavelengths were then estimated separately. These ratios were divided to form a red/infrared “ratio-of-ratio” value. The subjects were then asked to move their hands or head vigorously to simulate random motion encountered during ambulatory applications. This type of motion combined simultaneous left and right, front and back, as well as up and down movements to induce significant random motion artifacts.

The findings of Elgendi, M. Optimal Signal Quality Index for Photoplethysmogram signals. Bioengineering 2016, 3, 21; 1-15 are used below (see Elgendi, M. Optimal Signal Quality Index for Photoplethysmogram signals. Bioengineering 2016, 3, 21; 1-15, which is incorporated by reference herein in its entirety). Elgendi considered and based tests on eight signal quality indices (SQIs): perfusion, kurtosis, skewness, relative power, non-stationarity, zero crossing, entropy, and the matching of systolic wave detectors. The skewness index outperformed the other seven indices in differentiating between excellent PPG and acceptable, acceptable combined with unfit, and unfit recordings. Elgendi found that noisy data as characterized by random vigorous motion artifact, for example, had an absolute value for skewness of over 0.15. The skewness measure (in place of an accelerometer) was used to verify the motion artifact. The Fast Fourier Transform (FFT), morphological assessment and skewness evaluation of the motion signal (with absolute value of skewness>0.25) confirmed that the motion consisted of multiple frequency components distributed evenly to simulate vigorous, random motion in three dimensions.

The peak, mean and root mean square currents were then calculated again, this time with motion. The AC/DC ratio of the currents for the red and infrared wavelengths were then estimated again for motion separately. These red and infra-red ratios were further divided to give a red to infrared ratio for motion as follows:

$$\begin{matrix}Rnm = [ \frac{\frac{I red ac}{I red dc}}{\frac{I ir ac}{I ir dc}} ] no motion & ( 1 ) \\ Rm = [ \frac{\frac{I red ac}{I red dc}}{\frac{I ir ac}{I ir dc}} ] motion & ( 2 )\end{matrix}$$

Where, I is signal current, “red ac/dc” implies ac/dc signal from red LED and “it ac/dc” implies ac/dc signal from infrared LED.
The “red to infrared ratios” with motion were then subtracted from the “red to infrared ratios” without motion. This difference (designated as Δ) between the red and infrared ratios with and without motion was used as the key defining variable for the design of experiment model. Accordingly, Δ can be described by the following equation:

Δ=Rnm−Rm (3)

Under ideal conditions, where motion artifact is eliminated completely, Δ should be zero.

### Results

### Factor Determination

The initial experiments involved using a twelve run Plackett-Burman design with three factors and two levels on a single subject. The three factors used were sensor size (small and large), sensor weight (low and high) and relative motion against the skin as indicated by use of a skin adhesive (presence or absence). The global optimal response feature in the Plackett-Burmann design in Minitab® was used. This feature provides a desirability level for different combinations of the factors used in the design of experiments model. The desirability level is a metric for choosing a specific factor over the other due to its impact on the output. Skin adhesion showed a desirability level difference of 0.65, while sensor weight and sensor size change showed a desirability level difference of 0.19 and 0.04, respectively. Sensor size was found to have a non-significant impact on motion artifact, weight had a modest impact, and adhesion had a major impact. Therefore, ‘sensor weight’ and ‘relative motion controlled by a skin adhesive’ were chosen as the final two factors in addition to ‘placement’ and ‘contact force’ for their impact on the motion artifact signal.

### 3.2 Full Factorial Model Results

A full factorial design of experiments model was then developed in Minitab® to analyze the data with four factors, two levels and two repetitions on ten subjects. The model analyzed the data using an analysis of variance model, with sensor weight, placement, adhesion and force as the four input factors and all possible combinations of these factors. Table 1 shows the four factors and the two levels associated with each factor.

TABLE 1
Factors and Levels

| Factor | Level 1 | Level 2 |
|---|---|---|
| Sensor Weight | Low (8.44 g) | High (18.2 g) |
| Adhesion to skin | No | Yes |
| Placement | Forehead | Finger |
| Force (in Newtons) | Low (0.5 N) | High (5 N) |

In an ideal scenario, where the sensor and the design are completely immune to motion, the value of Δ, the key defining variable as explained in the experimental protocol, should be zero. However, due to various errors introduced by motion artifact, these Δ values can be positive or negative. These Δ readings were inserted in the ‘design of experiments’ full factorial model in Minitab® for a total vector of 320 readings (10 subjects using 2 repetitions in 16 runs gave a Δ vector of 320 readings consisting of 320 readings for R with motion minus 320 readings for R without motion).

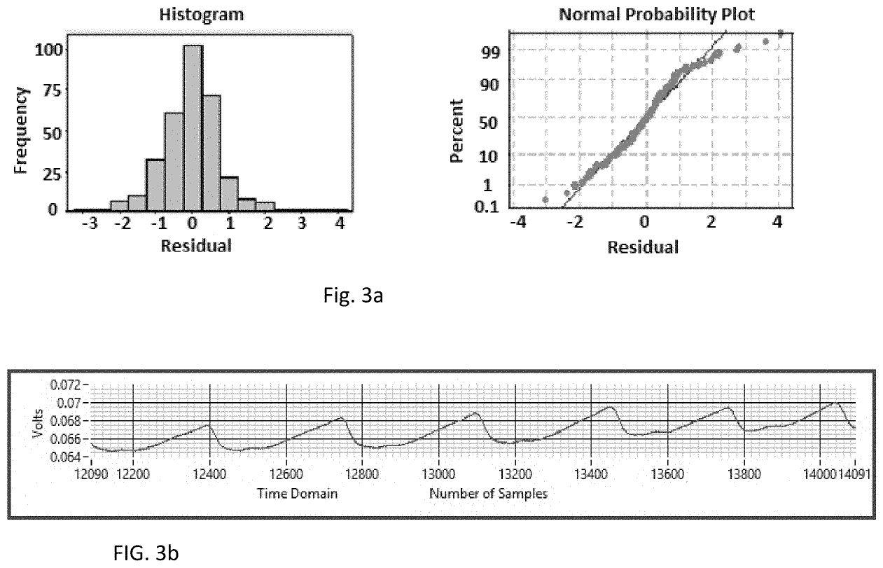

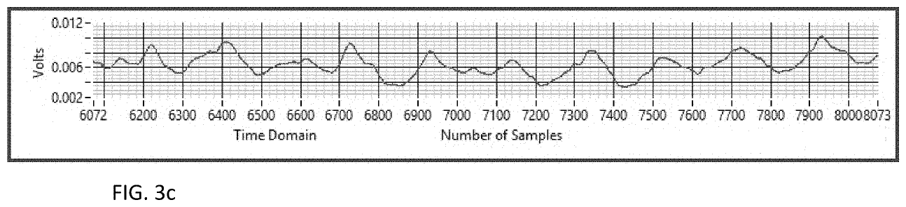

FIG. 3a shows the normality and independence of the data in terms of a histogram, and a normal probability plot of the residuals. The normal probability plot shows a linear fit for the residuals versus the percent distribution, while the histogram further shows the normal distribution of the residuals based on the frequency distribution. The residuals plot against fitted values did not show any non-uniformity. The graphs in FIG. 3a show that (the key assumptions of a multiple regression model) data were normal, independent, random and unbiased; hence, a high-level of confidence in the robustness of the prediction intervals and confidence levels in this chosen multiple regression model can be deduced. FIG. 3b shows the PPG signal without motion obtained in the time domain for one experimental run. FIG. 3c shows a similar PPG signal obtained in the time domain for one experimental run but with motion artifacts due to vigorous motion.

The full factorial design was implemented and analyzed in Minitab® using a multiple regression model with all the factors. Table 2a outlines the multiple regression equation obtained with the correlation coefficient. Eq (4) below shows that an optimal placement reduced Δ (the difference between a signal with no motion artifact and a signal mixed with motion artifact) by 0.1 or 10%, an optimal force (0.5N) reduced Δ by 0.13 or 13%, an optimal combination of weight (8.44 g) and adhesive (relative motion between the sensor and skin) reduced Δ by 0.16 or 16%, while an optimal combination of force and adhesion (relative motion) reduces Δ by 0.18 or 18% when the other variables are held constant. “Motion artifacts having a reduced effect,” as used herein, refers to reducing the value of Δ by more than 30%. Hence, the optimal values in the present experiment would include a sensor weight of 8.44 g, use of a hypoallergenic adhesive tape to secure the sensor, and a force of 0.5N on the forehead. This would have a strong cumulative effect in reducing the error rate and variation due to motion artifact.

Δ=−0.2Constant+0.1*Placement+0.13*Force+0.16*(Weight*Relative Motion)+0.18*(Force*Relative Motion) (4)

where,

### S (Sum of Squares)=0.80,

R-Sq (Coefficient of Determination)=14.32% and R-Sq (adjusted)=10.09%

TABLE 2a
Regression Equation and Analysis of Variance based on delta

| | | T | F | P- |
|---|---|---|---|---|
| Term | Coefficient | value | value | value |
| Constant | −0.20 | 4.48 | | 0.00 |
| Weight | −0.01 | 0.24 | 0.06 | 0.81 |
| Adhesion | −0.06 | 1.38 | 1.91 | 0.17 |
| Position (Placement) | 0.096 | 2.16 | 4.66 | 0.03 |
| Force | 0.13 | 2.87 | 8.22 | 0.004 |
| Weight*Adhesion | 0.16 | 3.60 | 12.97 | 0.000 |
| Weight*Position | 0.004 | 0.09 | 0.01 | 0.93 |
| Weight*Force | −0.02 | −0.5 | 0.25 | 0.62 |
| Adhesion*Position | 0.06 | 1.4 | 1.95 | 0.16 |
| Adhesion*Force | 0.18 | 4.00 | 16.02 | 0.00 |
| Position*Force | 0.026 | 0.59 | 0.35 | 0.56 |
| Weight*Adhesion*Position | −0.03 | 0.55 | 0.31 | 0.58 |
| Weight*Adhesion*Force | 0.08 | 1.75 | 3.06 | 0.08 |
| Weight*Position*Force | 0.001 | 0.02 | 0.00 | 0.98 |
| Adhesion*Position*Force | −0.04 | 0.85 | 0.72 | 0.40 |
| Weight*Adhesion*Position*Force | 0.026 | 0.58 | 0.34 | 0.56 |

The bold values in Table 2A indicate factors that have a significant effect on the dependent variable at the 95% confidence level, while the italicized values include factors that are significant at the 80% confidence level. Table 2a outlines the F statistic and p value of each factor or factor combination. (F values are values of the F statistic, which is a ratio of two variances. See, for example, http://blog.minitab.com/blog/adventures-in-statistics-2/understanding-analysis-of-variance-anova-and-the-f-test. The p values are used to determine statistical significance in a hypothesis test. See, for example, http://blog.minitab.com/blog/adventures-in-statistics-2/how-to-correctly-interpret-p-values.) Assuming a type 1 error (alpha) of 0.05, a p-value less than 0.05 signifies that factor or factor combination has a significant effect on the overall delta. Table 2b shows the standard error and least square means of the significant factors. The standard error determines the amount by which the factor or factor interaction reduces the residual error between the estimated value (from the model) and actual value. A lower negative number indicates that the single factor or two factor combination reduces the standard error by a higher value and is preferable. The least square mean is an adjusted mean that is sensitive to other co-variates in the regression model and is a better estimate of the actual mean. The low values of the least square means indicate that the model estimates for the variable delta are close to zero as expected.

TABLE 2b
Standard Error and Least Squares Means

| | | |
|---|---|---|
| Term | Standard Error | Least Square Means |

| Weight | | | |
|---|---|---|---|
| 8.44 g | | −0.21 | 0.063 |
| 18.20 g | | −0.19 | 0.063 |
| Adhesion | | | |
| No | | −0.14 | 0.063 |
| Yes | | −0.26 | 0.063 |
| Position | | | |
| Finger | | −0.10 | 0.063 |
| Forehead | | −0.30 | 0.063 |
| Force | | | |
| High | | −0.07 | 0.063 |
| Low | | −0.33 | 0.063 |
| Weight*Adhesion | | | |
| 8.44 g | No | 0.01 | 0.089 |
| 8.44 g | Yes | −0.43 | 0.089 |
| 18.20 | No | −0.29 | 0.089 |
| 18.20 | Yes | −0.09 | 0.089 |
| Adhesion*Force | | | |
| No | High | 0.17 | 0.089 |
| No | Low | −0.44 | 0.089 |
| Yes | High | −0.31 | 0.089 |
| Yes | Low | −0.21 | 0.089 |

Based on the experiments, a range of values of parameters for design that result in a reduced Δ (the difference between a signal with no motion artifact and a signal mixed with motion artifact) is obtained, and the results are shown in Table 3 below,

TABLE 3

| Parameter | Optimal | | Lower Limit | | Upper Limit | |
|---|---|---|---|---|---|---|
| Red Frequency | 660 | nm | 640 | | 680 | nm |
| Infrared Frequency | 905 | nm | 885 | nm | 925 | nm |
| LED Current | 21 | mA | 17 | mA | 25 | mA |
| Force/area | 1.2N/cm\hat{ }2 (12 KPa) | | 0.3 N/cm\hat{ }2 (3 kPa) | | 1.5 N/cm\hat{ }2 (15 kPa) | |
| Relative Motion (Bonding | 15N/cm\hat{ }2 | | 10 N/cm\hat{ }2 | | 20 N/cm\hat{ }2 | |
| pressure) | | | | | | |
| Weight | 8.44 | g | 6 | g | 12 | g |

| | | | |
|---|---|---|---|
| Placement | Forehead- Left | Wrist depending | Finger |
| | temple to Right | on best signal per | depending on |
| | Temple depending | criteria below | best signal per |
| | on best signal per | (signal quality | criteria below |
| | criteria below | index) | (signal quality |
| | (signal quality | | index). |
| | index). | | |
| Waveform Quality -Signal to | 60 | 40 | 80 |
| noise ratio(NSQI = | | | |
| σ\hat{ }2signal/σ\hat{ }2noise) | | | |
| Waveform Quality -Skewness(S = | 0 | −0.35 | 0.35 |
| σ\hat{ }2signal/σ\hat{ }2noise) | | | |
| Waveform Quality - Kurtosis - | 2.05 | 1.8 | 2.2 |

Selecting placement and the two most important terms in Table 2a (contact force, and a sensor to skin adhesion and contact force combination), a reflective PPG sensor in which motion artifacts have a reduced effect is placed on one of the forehead, the finger or the wrist of the subject according to a predetermined range of a signal quality index and has a force per unit area between 0.3 N/cm² and 1.5 N/cm² and a bonding pressure, for sensor to skin adhesion, between 10 N/cm² and 20 N/cm². When the three most important terms in Table 2a (contact force, a sensor to skin adhesion and contact force combination and a sensor weight and sensor to skin adhesion combination) are selected, the reflective PPG sensor in which motion artifacts have a reduced effect is placed on one of the forehead, the finger or the wrist of the subject according to a predetermined range of a signal quality index and has a force per unit area between 0.3 N/cm² and 1.5 N/cm² and a bonding pressure, for sensor to skin adhesion, between 10 N/cm² and 20 N/cm² and a weight between 6 g and 12 g. In one instance, a location of the reflective PPG sensor is on the forehead and is selected such that a signal quality index is in predetermined range of a signal quality index values. Three measures of signal quality index are given in Table 3-signal-to-noise ratio, skewness and kurtosis. Eight measures of signal quality index were used by Elgendi of which five important measures included:

(1) Perfusion (most commonly used)—is the difference of the amount of light absorbed through the pulse of when light is transmitted through the finger, which can be defined as follows:

P_{SQI}=[(yₘₐₓ−yₘᵢₙ)/|x̅|]×100;

(2) Skewness—is a measure of the symmetry (or the lack of it) of a probability distribution, which is defined as:

$${S}_{SQI} = 1 / N {\sum }_{i = 1}^{N} {[ {x}_{i} - {\hat{μ}}_{x} / σ ]}^{3} ;$$

(3) Kurtosis—represents a heavy tail and peakedness or a light tail and flatness of a distribution relative to the normal distribution, which is defined as:

$${K}_{SQI} = 1 / N {\sum }_{i = 1}^{N} {[ {x}_{i} - {\hat{μ}}_{x} / σ ]}^{4}$$

where μₓ and σ are the empirical estimate of the mean and standard deviation of xᵢ, respectively; and N is the number of samples in the PPG signal;
(4) Entropy—quantifies how much the probability density function (PDF) of the signal differs from a uniform distribution is defined as:

$${E}_{SQI} = - {\sum }_{n = 1}^{N} {x [ n ]}^{2} {log}_{e} ( {x [ n ]}^{2} )$$

where x[n] is the raw PPG signal and N is the number of data points;
(5) Signal-to-noise ratio.
If the teachings of Elgendi are followed, the skewness is selected as the principal signal quality index with perfusion as the initial filtering index.

### Results

As seen in Table 2a, force (p=0.004) and placement (p=0.03) had a significant effect on motion artifacts. Weight (p=0.81), by itself did not have a significant effect on motion artifact, and adhesion had some effect (p=0.17), but the ‘two-factor combination’ of weight coupled with adhesion (p<0.001) as well as the ‘two-factor combination’ of adhesion coupled with force (p<0.001) further increased the effect on motion artifact. A lower weight can negate the effect of adhesion or contact force during motion due to a lower inertia and, therefore, an optimal sensor weight with adhesion can significantly reduce the effect of motion artifact. It should be noted that the three-factor combination of adhesion, weight and force showed marginal significance (p=0.08) and seems to have some effect on motion artifact. This supports the concept that the most optimal sensor variables include a sensor weighing 8.44 g, applied with a hypoallergenic adhesive tape having a bonding pressure between 10 N/cm\hat{ }2 and 20 N/cm\hat{ }2 and with a force of 0.5 N on the forehead.

A sensor with a sensor area of 0.42 cm² was used, so a contact force of 0.5N corresponds to a contact pressure of 12 KPa. The present results showed that a contact force of 0.5N used with the sensor of these teachings would be ideal for a forehead sensor. Unlike the wrist, a contact pressure with an adhesive and a headband would not be a constraint for a forehead-based sensor. These teachings also demonstrate that motion artifact can be minimized by a proper selection of these other sensor variables, removing motion artifact as a constraint in the development of a commercial product. The sensor of these teachings can be used for a variety of measurements including, but not limited to, an SpO₂ measurement as well as the associated pulse rate measurement derived from the motion artifact reduced “SpO₂ waveform”.

The regression model of these teachings gave a correlation value of 0.14, which indicates that only 14% of the variation can be explained by this model. Clarke (in Clarke, Geoffrey. Signal Quality Analysis in Pulse Oximetry: Modelling and Detection of Motion Artifact. Masters Thesis, Ottawa-Carleton Institute for Biomedical Engineering, Carleton University, Ottawa University, Ottawa, Ontario, 2015) observed high variation due to high motion artifacts. This was due to the various other sources of error that made the data noisy. This may have been specifically due to variation in the angles of reflected light captured by the photodetector among different subjects, differing optical extinction coefficients for deoxygenated hemoglobin (Hb) and oxygenated hemoglobin (HbO₂) due to differing physiology among subjects, electronic noise associated with manufacturing tolerances in the frequencies of red and infrared light emitted by the LEDs. In addition, optical noise is also introduced due to varying pathlengths and differing time-variant absorption by the moving venous blood among subjects as well as air gaps between the sensor and the skin due to various levels of random motion in different trials. Although only 10 subjects contributed data to this study, there were a total of 320 data points for analysis, adding to the overall statistical power. A sample size of 10 subjects provided adequate statistical power (>90%) under the full factorial design at a two-sided alpha of 5% and the observed differences between the factor levels. This was based on the assumption that the difference in readings with and without motion can be as small as 0.1 in magnitude and with a standard error as large as 0.9. Despite the high variation due to noise in the data, the data showed a high-level of normality, and the effect of the factors chosen in terms of the confidence level became even more pronounced. A strong ‘p’ value seen in some factors or factor combinations despite this lower value for “r” indicates a strong influence of these factors or factor interactions on motion artifact.

These teachings revealed that applied force, PPG quality based on placement, relative sensor motion with respect to the skin (adhesion of the sensor to the skin) and sensor weight are all important variables in designing a robust reflectance-based pulse oximeter sensor for suppressing motion artifact from the PPG. The applied force and quality of the PPG obtained based on placement had a significant effect on motion artifact, while relative motion based on adhesion had an important effect. Sensor weight by itself had a minimal effect, but when combined with adhesion had a significant effect on suppressing motion artifact. Similarly, contact force combined with adhesion had a significant effect on suppressing motion artifact.

The reflectance-mode PPG sensor design of these teachings for minimizing motion artifact is one that has a unique combination of these sensor variables that include sensor weight, a hypoallergenic adhesive tape to restrict relative motion between the sensor and the skin, placement at a body location with a high amplitude for PPG pulse magnitude based on signal quality index or morphology algorithms and a caressing, but firm, contact force.

The use of the word “about,” avoids a strict numerical boundary to the specified parameter. The word “about,” as used herein, refers to the uncertainty in the value that comes from the uncertainty in measurements of that value. Such uncertainty is typical of every measurement equipment and measurement method, and ranges up to 10 to 15%.

Although the invention has been described with respect to various embodiments, it should be realized these teachings are also capable of a wide variety of further and other embodiments within the spirit and scope of the appended claims.

What is claimed is:

1. A reflective PPG sensor where motion artifacts have a reduced effect, the reflective PPG sensor being configured to be placed on one of a forehead, a finger or a wrist of a subject on a location selected according to a predetermined range of a signal quality index, the reflective PPG sensor having:
a force per unit area between 0.3 N/cm² and 1.5 N/cm²; and,
a bonding pressure, for sensor to skin adhesion, between 10 N/cm² and 20 N/cm².

2. The reflective PPG sensor of claim 1 further having a weight between 6 g and 12 g.

3. The reflective PPG sensor of claim 1 wherein the force per unit area is about 1.2 N/cm² and the bonding pressure is about 15 N/cm².

4. The reflective PPG sensor of claim 2 wherein the force per unit area is about 1.2 N/cm², the bonding pressure is about 15 N/cm² and the weight is about 8.44 g.

5. The reflective PPG sensor of claim 1 wherein a location of the reflective PPG sensor is on the forehead and is selected such that the signal quality index is in the predetermined range.

6. The reflective PPG sensor of claim 5 wherein the signal quality index is skewness.

7. The reflective PPG sensor of claim 5 wherein the signal quality index is perfusion.

8. The reflective PPG sensor of claim 2 wherein a location of the reflective PPG sensor on the forehead, wrist or finger is selected such that a signal quality index is in the predetermined range.

9. The reflective PPG sensor of claim 8 wherein the signal quality index is skewness.

10. The reflective PPG sensor of claim 8 wherein the signal quality index is perfusion.

11. A method for selecting a reflective PPG sensor in which motion artifacts have a reduced effect, the method comprising:
selecting a placement of the reflective PPG sensor on a subject,
selecting a contact force, and
selecting a sensor to skin adhesion and contact force combination, such that motion artifacts have a reduced effect.

12. The method of claim 11 further comprising selecting a sensor weight and sensor to skin adhesion combination such that motion artifacts have a reduced effect.

13. The method of claim 12 wherein the placement of the reflective PPG sensor on the subject, the contact force, the sensor to skin adhesion, and the sensor weight are selected using a full factorial design of experiments with four factors.

14. The method of claim 12 wherein the placement of the reflective PPG sensor on the subject, the contact force, the sensor to skin adhesion, and the sensor weight are selected using a fractional factorial design of experiments with four factors.

15. The method of claim 11 wherein selecting the placement of the reflective PPG sensor on the subject comprises selecting placing the reflective PPG sensor on one of a forehead, a finger or a wrist of the subject; and wherein the method further comprises selecting a location of the reflective PPG sensor such that a signal quality index is in a-predetermined range of values.

16. The method of claim 15 wherein the signal quality index is skewness.

17. The method of claim 15 wherein the signal quality index is perfusion.

18. The method of claim 12 wherein selecting the placement of the reflective PPG sensor on the subject comprises placing the reflective PPG sensor on one of a forehead, a finger or a wrist of a subject on a location selected according to a predetermined range of a signal quality index; and wherein sensor force per unit area is between 0.3 N/cm² and 1.5 N/cm², a bonding pressure, for sensor to skin adhesion, is between 10 N/cm² and 20 N/cm², and a sensor weight between 6 g and 12 g.

19. The method of claim 18 wherein the sensor force per unit area is about 1.2 N/cm², the bonding pressure is 15 N/cm² and the sensor weight is about 8.44 g.
